(19) United States
(12) Patent Application Publication
(10) Pub. No.: US 2020/0179711 A1
(43) Pub. Date: Jun. 11, 2020

(54) PHOTODYNAMIC THERAPY LIGHT IRRADIATION DEVICE

(71) Applicants: PUBLIC UNIVERSITY CORPORATION NAGOYA CITY UNIVERSITY, Nagoya-city, Aichi (JP); Ushio Denki Kabushiki Kaisha, Tokyo (JP); SHARP KABUSHIKI KAISHA, Osaka (JP)

(72) Inventors: Akimichi MORITA, Aichi (JP); Hideyuki MASUDA, Tokyo (JP); Makoto KIMURA, Tokyo (JP); Katsuji IGUCHI, Osaka (JP); Jun MORI, Osaka (JP)

(73) Assignees: PUBLIC UNIVERSITY CORPORATION NAGOYA CITY UNIVERSITY, Nagoya-city, Aichi (JP); Ushio Denki Kabushiki Kaisha, Tokyo (JP); SHARP KABUSHIKI KAISHA, Osaka (JP)

(21) Appl. No.: 16/623,310

(22) PCT Filed: Jun. 20, 2017
(86) PCT No.: PCT/JP2017/022735

§ 371 Date: Dec. 16, 2019

(51) Int. Cl. A61N 5/06; A61K 41/00

(52) U.S. Cl. CPC A61N 5/062; A61N 2005/0663; A61N 2005/0652; A61K 41/0057

(57) ABSTRACT

An object of the present invention is to provide a photodynamic therapy light irradiation device for irradiating two kinds of light in different wavelength ranges to an irradiated surface, the device being capable of irradiating the light with uniform illuminance to the irradiated surface even having an uneven shape and obtaining a spectral distribution of high uniformity on the entire irradiated surface.

A photodynamic therapy light irradiation device of the present invention is characterized by including: a light source unit having one or more LED elements that emit first light having a peak wavelength within a range of wavelengths of not shorter than 400 nm to not longer than 420 nm disposed on a flexible substrate; and a fluorescent plate configured to transmit a part of the first light from the light source unit, and to convert another part thereof into second light having a wavelength of not shorter than 500 nm to not longer than 520 nm and thereby emit the second light.

### TECHNICAL FIELD

The present invention relates to a photodynamic therapy light irradiation device.

### BACKGROUND ART

Conventionally, a photodynamic therapy (hereinafter also referred to as “PDT”) has been known as one of therapies using light. The PDT is a therapy in which, using properties of a photosensitizer having affinity with a lesion (lesioned abnormal tissue) in the living body, specifically, using properties of a photosensitizer being specifically accumulated in a lesion, the photosensitizer or a precursor thereof is administered to the living body, and then the photosensitizer (including the photosensitizer synthesized from the precursor of the photosensitizer in the living body) is irradiated with light (visible light) to selectively destroy only the lesioned abnormal tissue using a reactive oxygen species produced in the tissue. Such a PDT is expected as a minimum invasive therapy. Further, in recent years, the PDT has been widely used in the field of dermatology for therapies of, for example, neoplastic lesions such as solar keratosis, Bowen disease, Paget disease and basal cell carcinoma, severe acne vulgaris, sebaceous hyperplasia and intractable warts.

In a photodynamic therapy light irradiation device for performing such a PDT (hereinafter also referred to as “PDT light irradiation device”), a laser light source having a wavelength of 600 to 700 nm and a lamp light source such as a xenon lamp and a metal halide lamp are used.

In recent years, the PDT light irradiation device using an LED element as a light source instead of the laser light source and the lamp light source has been proposed (see Patent Literature 1). This PDT light irradiation device includes a light source unit in which a first LED element having a peak wavelength in a wavelength of 400 to 420 nm and a second LED element having a peak wavelength in a wavelength of 500 to 520 nm are alternately arranged in a lattice pattern. Further, when the first LED element and the second LED element are both turned on, the same irradiated site is irradiated with light from the first LED element and light from the second LED element.

It is expected that, according to such a PDT device, an irradiation amount (integrated light amount) required for the therapy can be reduced as compared with a case where the light from the first LED element and the light from the second LED element are each separately irradiated to the site, making it possible to shorten an irradiation time required for the therapy.

### CITATION LIST

### Patent Literature

Patent Literature 1: Japanese Patent Application Laid-Open No. 2017-6454

### SUMMARY OF INVENTION

### Technical Problem

However, the PDT light irradiation device described above has the following problems.

(1) A skin disease is known to often develop in the face or the neck due to the exposure to sunlight. Further, the face, for example, a surface of the nose or the cheek, is not formed in a flat plane but in an uneven shape.

On the other hand, in the above-described PDT light irradiation device having a configuration in which a plurality of LED elements are arranged, the LED element having a large divergence angle of light (for example, a full divergence angle of 135°) is used for uniformalizing an illuminance distribution on an irradiated surface. However, in the LED element having a large divergence angle, the illuminance largely depends on an irradiation distance, and thus, when the lesion has an uneven shape as is the case for the nose or the cheek, the illuminance on a recess surface is significantly lower than that on a protrusion surface, resulting in so-called therapy unevenness. Further, in a case where the LED element having a small divergence angle of light (for example, a full divergence angle of 30°) is used, the dependency of the illuminance on the irradiation distance becomes small; however, the uniformity of the illuminance distribution on the irradiated surface is low, also resulting in the therapy unevenness.

(2) In the PDT light irradiation device described above, the first LED element and the second LED element are alternately arranged. Thus, when the PDT light irradiation device is arranged closely to the irradiated surface, there are a region where the illuminance of the light from the first LED element is high and a region where the illuminance of the light from the second LED element is high on the irradiated surface. Thus, the uniformity of the spectral distribution is reduced on the irradiated surface. Further, when the PDT light irradiation device is arranged largely apart from the irradiated surface, the illuminance on the irradiated surface is reduced, requiring a long irradiation time for achieving a sufficient therapy.

(3) In the above-described PDT light irradiation device, the second LED element has a peak wavelength in a wavelength of 500 to 520 nm. However, such an LED element with high illuminance has not been put to practical use. Thus, configuring the above-described PDT light irradiation device requires attenuating light from the first LED element by, for example, a filter. This increases the number of parts in the PDT light irradiation device, thereby increasing the production cost of the PDT light irradiation device. Further, it becomes difficult to irradiate light with high illuminance to the irradiated surface, requiring a long irradiation time for achieving a sufficient therapy.

An object of the present invention is to provide a photodynamic therapy light irradiation device for irradiating two kinds of light in different wavelength ranges to an irradiated surface, the device being capable of irradiating the light with uniform illuminance to the irradiated surface even having an uneven shape and obtaining a spectral distribution of high uniformity on the entire irradiated surface.

### Solution to Problem

A photodynamic therapy light irradiation device of the present invention is characterized by including:

a light source unit having one or more LED elements that emit first light having a peak wavelength within a range of wavelengths of not shorter than 400 nm to not longer than 420 nm disposed on a flexible substrate; and

a fluorescent plate configured to transmit a part of the first light from the light source unit, and to convert another part thereof into second light having a wavelength of not shorter than 500 nm to not longer than 520 nm and thereby emit the second light.

In the photodynamic therapy light irradiation device of the present invention, it is preferable that the light source unit includes a plurality of the LED elements.

Further, it is preferable that the fluorescent plate is disposed such that the first light and the second light are superimposed on an irradiated surface.

Further, in the photodynamic therapy light irradiation device of the present invention, it is preferable that the light irradiated from the fluorescent plate to the irradiated surface satisfies the following formula (1) where an irradiance integral value of light within a range of wavelengths of not shorter than 350 nm to not longer than 455 nm on the irradiated surface is defined as IA and an irradiance integral value of light within a range of longer than 455 nm to not longer than 650 nm on the irradiated surface is defined as IB. Note that the term “illuminance” in the following description means, unless otherwise specified, the total of the IA and the IB.

IA/IB=0.2 to 5 Formula (1)

In the photodynamic therapy light irradiation device described above, it is more preferable that IA/IB in the aforementioned formula (1) is 1 to 1.8.

Further, in the photodynamic therapy light irradiation device of the present invention, it is preferable that:

the light source unit includes a wall material formed so as to surround a region where the LED element is disposed on the flexible substrate and a protective resin layer formed so as to cover the LED element in the region where the LED element is disposed, the region being surrounded by the wall material; and

the fluorescent plate is disposed so as to cover upper surfaces of the protective resin layer and the wall material.

Further, it is preferable that a contact member configured to have transparency and be brought into contact with the irradiated surface is provided so as to cover at least the fluorescent plate.

Further, in the photodynamic therapy light irradiation device of the present invention, it is preferable that the fluorescent plate includes Ba₂SiO₄:Eu as a fluorescent material.

### Advantageous Effects of Invention

The photodynamic therapy light irradiation device of the present invention is capable of irradiating the light with uniform illuminance to an irradiated surface even having an uneven shape and obtaining a spectral distribution of high uniformity on the entire irradiated surface.

### BRIEF DESCRIPTION OF DRAWINGS

FIG. 1 is an explanatory cross-sectional view illustrating an example of a configuration of a photodynamic therapy light irradiation device of the present invention.

FIG. 2 is an explanatory diagram illustrating an arrangement state of LED elements on a surface of a flexible substrate in the photodynamic therapy light irradiation device shown in FIG. 1.

FIG. 3 is a diagram illustrating an optical spectrum of light emitted from the photodynamic therapy light irradiation device according to Example.

### DESCRIPTION OF EMBODIMENTS

Embodiments of the present invention will be described below in detail.

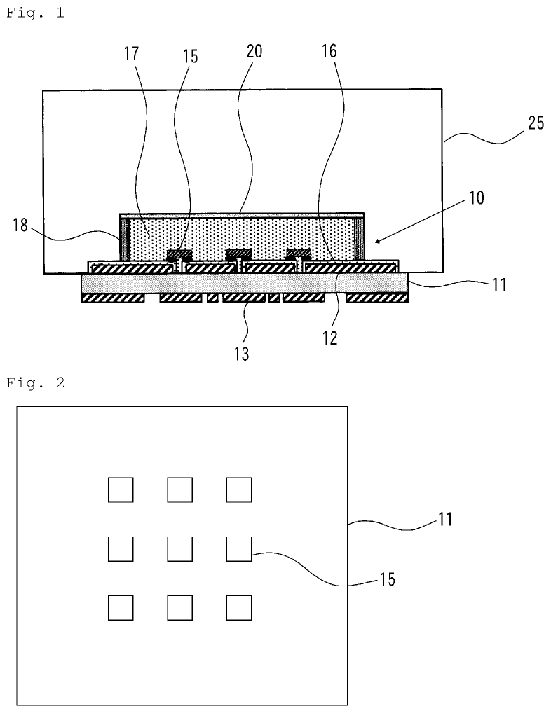

FIG. 1 is an explanatory cross-sectional view illustrating an example of a configuration of a photodynamic therapy light irradiation device of the present invention.

This PDT light irradiation device is configured to perform a photodynamic therapy in which a substance to be administered to the living body including a photosensitizer or a precursor of the photosensitizer is administered to the living body, and then light is irradiated to the photosensitizer (including the photosensitizer synthesized from the precursor of the photosensitizer in the living body) accumulated in a lesion (lesioned abnormal tissue).

As the substance to be administered to the living body, a compound that is reacted in the living body as needed and accumulated as a porphyrin compound in the lesion, or the like is used.

As a specific example of the substance to be administered to the living body, may be mentioned δ-aminolevulinic acid (5-ALA). The δ-aminolevulinic acid is a precursor of a photosensitizer, and protoporphyrin IX (PpIX) synthesized through an enzymatic reaction functions as the photosensitizer.

The PDT light irradiation device shown in FIG. 1 includes a light source unit 10 including a plurality of LED elements 15 and a fluorescent plate 20 arranged on the light source unit 10. In the PDT device in this example, a contact member 25 configured to have transparency and be brought into contact with an irradiated surface, that is, a lesion is provided so as to cover a surface of a flexible substrate 11 described below and the fluorescent plate 20.

The light source unit 10 includes a flexible substrate 11 on which wiring portions 12 and 13 made of, for example, copper are formed on the front surface (upper surface in FIG. 1) and the back surface, respectively. On the surface of the flexible substrate 11, the plurality of LED elements 15 are mounted and arranged by, for example, a flip-chip mounting method. In the flip-chip mounting method, a thermal history of 200° C. or higher is applied to the wiring portion 12, a light reflecting film 16, and the like.

Mounting of the LED elements 15 is preferably performed by, but not limited to, a flip-chip mounting method so that the light source unit 10 can be bent along shapes of the lesion and its surrounding. Using wire bonding mounting may cause disconnection of a wire when being bent along the shapes of the lesion and the like.

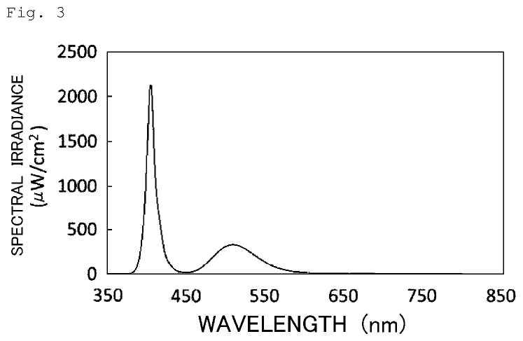

As shown in FIG. 2, the respective LED elements 15 in the light source unit 10 are arranged in a lattice pattern on the surface of the flexible substrate 11 at a predetermined arrangement pitch (center distance). In the example illustrated in the figure, the LED elements 15 are arranged in a lattice form of three rows and three columns.

The light reflecting film 16 is formed on the surface of the wiring portion 12. By providing such a light reflecting film 16, light from the LED elements 15 and the fluorescent plate 20 can be reflected toward the lesion. In addition, it is possible to expect the effect that the light irradiated toward the lesion and reflected back from the surface of the lesion and its surrounding is reflected again toward the lesion. It is also possible to prevent light leakage from the back surface of the flexible substrate 11.

In addition, a rectangular frame-shaped wall material 18 is formed on the surface of the flexible substrate 11 so as to surround a region where the LED elements 15 are disposed. The shape of the wall material 18 is not limited to a rectangle, but may be a circle, an ellipse, or a polygon. A material having a shape suitable for the shape of the lesion is used as the wall material 18. The height of the wall material 18 is greater than the height of the LED elements 15 from the flexible substrate 11. A protective resin layer 17 is provided in a region surrounded by the wall material 18 where the LED elements 15 are disposed so as to cover each of the LED elements 15 and the wiring portion 12. The thickness of the protective resin layer 17 from the flexible substrate 11 is equal to the height of the wall material 18. The fluorescent plate 20 is disposed so as to cover the upper surface of the protective resin layer 17 and the upper surface of the wall material 18.

According to such a configuration, it is possible to prevent the light from the LED elements 15 from being irradiated to the lesion without passing through the fluorescent plate 20. Further, when the contact member 25 is disposed on the fluorescent plate 20, a pressure is applied to the fluorescent plate 20; however, since the pressure is dispersed in the protective resin layer 17 and the wall material 18, it is possible to prevent the wiring failure of the LED elements 15 from occurring.

The flexible substrate 11 is a flexible insulating substrate made of a resin material, and is formed from an insulating film such as polyimide. However, the material of the flexible substrate 11 is not limited to polyimide, and any material can be used as long as it is an insulating material and has the required mechanical strength and flexibility. In addition to the polyimide resin film, for example, a film such as a fluororesin, a silicone resin, or a polyethylene terephthalate resin can be used as the flexible substrate 11. Moreover, as the flexible substrate 11, a highly reflective resin film in which a resin (white resin, white resist, etc.) containing a white pigment is applied to the surface of these films, a highly reflective film in which a white pigment is mixed, a liquid crystal polymer film and the like can be used.

Further, the thickness of the flexible substrate 11 is, for example, 25 to 200 μm. If the thickness of the flexible substrate 11 is too small, it may be difficult to obtain a required mechanical strength. On the other hand, if the thickness of the flexible substrate 11 is too large, it may be difficult to obtain necessary flexibility. That is, mechanical strength and flexibility are in a trade-off relationship regarding the thickness of the flexible substrate 11, and an optimum value exists. It is more preferable that the thickness of the flexible substrate 11 is 40 to 100 μm.

The size of the flexible substrate 11 is not particularly limited. The flexible substrate 11 is only required to have a size covering the lesion. However, when the flexible substrate 11 is formed in a size that allows light irradiation in a state in which surfaces of the lesion and its surrounding are covered with the light source unit 10, it becomes possible to reduce a constraint of a patient and minimize a burden of the patient.

The PDT light irradiation device of the present invention is suitably used for a local lesion having a relatively small area of about several centimeters. In such a PDT light irradiation device, it is preferable that the flexible substrate 11 is formed in a size corresponding to the local lesion.

As the LED element 15, those that emit the first light having a peak wavelength within a range of wavelengths of not shorter than 400 nm to not longer than 420 nm is used. The LED element 15 in this example emits light having a peak wavelength at a wavelength of 405 nm.

A substantially square shape can be adopted as the planar shape of each LED element 15. The planar shape is not limited to a substantially square shape.

The length of one side of the LED element 15 is, for example, 0.6 to 1.5 mm. A photodynamic therapy generally requires an energy density of 50 to 100 J/cm². For example, when a therapy is performed under a condition where the light irradiation time is 15 minutes, an average irradiance of 55.6 to 111 mW/cm² is required. If the length of one side of the LED element 15 is too small, that is, if the area of the LED element 15 is too small, the maximum current that is allowed to pass through the LED element 15 becomes small, and thus it may be difficult to secure the average irradiance described above. On the other hand, if the length of one side of the LED element 15 is too large, the arrangement pitch of the LED elements 15 must be increased from the viewpoint of in-plane uniformity of the average irradiance of the light source unit 10, and there arises a problem in which the light source unit 10 becomes large in area.

In this embodiment, the LED element 15 is substantially square, the length of one side thereof is 1 mm, and the thickness is 0.15 mm.

The arrangement pitch of the LED elements 15 depends on the dimensions of the LED elements 15, but is preferably 3 to 15 mm. In the present embodiment, the arrangement average pitch of the LED elements 15 is about 5 to 10 mm.

As a material constituting the light reflecting film 16, silver, aluminum, a resin containing a white pigment, or the like can be used. When the LED element 15 is flip-mounted, a heat history of not lower than 200° C. is applied to the light reflecting film 16 as described above. Therefore, the light reflecting film 16 needs to have heat resistance at this temperature or higher. From this viewpoint, the material constituting the light reflecting film 16 is preferably silver or aluminum.

In the following, the term “total light reflectance” is used, but it is not the reflectance of the mirror-surface reflection, but a ratio of the light energy obtained by integrating all the diffusely reflected light relative to the energy of the incident light. It is preferable that the light reflecting film 16 is constituted by a reflective material having a total light reflectance of not less than 80% (hereinafter referred to as “high reflectance material”), in particular, a high reflectance material having a total light reflectance of not less than 90%. By providing such a light reflecting film 16, light from the LED elements 15 and the fluorescent plate 20 can be efficiently reflected toward the lesion. In addition, it is possible to expect the effect that the light irradiated toward the lesion and reflected back from the surface of the lesion and its surrounding is reflected again toward the lesion. It is also possible to prevent light leakage from the back surface of the flexible substrate 11.

The thickness of the light reflecting film 16 is, for example, 3 to 10 μm. In the present embodiment, the light reflecting film 16 having a thickness of 5 μm is formed by silver plating.

It is preferable that the protective resin layer 17 is transparent. Specifically, it is preferable that the transmittance of the protective resin layer 17 is not less than 80% with respect to the first light and the second light. With this configuration, the power consumption of the light source unit 10 can be reduced, and at the same time, the amount of heat generated by the light source unit 10 can be reduced.

It is preferable that the protective resin layer 17 is flexible.

As the material constituting the protective resin layer 17, a silicone resin, an epoxy resin, or the like can be used.

The thickness of the protective resin layer 17 is, for example, 0.5 to 1 mm. In the present embodiment, the thickness of the protective resin layer 17 is about 0.8 mm.

The wall material 18 is formed of a silicone resin. The wall material 18, which is formed of a light reflecting material, has light reflectivity. With this configuration, the light from the LED elements 15 can be reflected by the wall material 18, so that the light can be extracted through the protective resin layer 17. Furthermore, since the wall material 18 is formed so as to be in contact with the fluorescent plate 20, that is, the entire surface of the protective resin layer 17 is covered with the fluorescent plate 20, the light emitted from the LED elements 15 can be prevented from entering the lesion without passing through the fluorescent plate 20. Thereby, the in-plane uniformity of the value of IA/IB described later is improved.

Moreover, since the protective resin layer 17 having a uniform thickness can be easily formed by forming the wall material 18, it is possible to reduce manufacturing defects due to the LED elements 15 being exposed.

The fluorescent plate 20 transmits a part of the first light from the light source unit 10 and converts another part of the first light into second light having a wavelength of not shorter than 500 nm to not longer than 520 nm and emits the second light. As such a fluorescent plate 20, a plate in which a fluorescent material is dispersed in a transparent substrate can be used.

Further, the thickness of the fluorescent plate 20 is, for example, 0.3 to 1 mm.

As the transparent substrate forming the fluorescent plate 20, a flexible resin such as a silicone resin, an epoxy resin, or a styrene-based elastomer can be used. In this embodiment, a silicone resin is used.

As the fluorescent material, one that receives the first light having a peak wavelength within a range of wavelengths of not shorter than 400 nm to not longer than 420 nm and emits the second light having a wavelength of not shorter than 500 nm to not longer than 520 nm as fluorescence is used. As specific examples of such a fluorescent material, may be mentioned BaSi₂(O,Cl)₂N₂:Eu, (Ba,Sr)MgAl₁₀O₁₇:(Eu,Mn), BaMgAl₁₀O₁₇:(Eu,Mn), (Ba,Sr)₂SiO₄:Eu, Ba₂SiO₄:Eu, SrAl₂O₄:Eu, (Sr,Al)₆(O,N)₈:Eu and the like.

When δ-aminolevulinic acid (5-ALA) is used as the substance to be administered to the living body, protoporphyrin IX (PpIX) functions as a photosensitizer. Patent Literature 1 discloses that the absorption spectrum of PpIX has absorption peaks at a wavelength of 410 nm, a wavelength of 510 nm, a wavelength of 545 nm, a wavelength of 580 nm, and a wavelength of 630 nm. For this reason, Ba₂SiO₄:Eu having a peak in the vicinity of 510 nm which is the second absorption wavelength of PpIX is most suitable as the fluorescent material. Further, since the emission of Ba₂SiO₄:Eu has a wide half-value width of 64 nm, light with a wavelength of 545 nm, which is the third absorption wavelength of PpIX, or the vicinity thereof is expected to be absorbed by PpIX.

The content ratio of the fluorescent material is preferably a ratio of 1 to 20 parts by mass per 100 parts by mass of the transparent resin, although it depends on the intensity of light emitted from the LED elements 15 and the thickness of the fluorescent plate 20.

If the content ratio of the fluorescent material is too small, the in-plane distribution of the content ratio of the fluorescent material contained in the fluorescent plate 20 becomes large. On the other hand, if the content ratio of the fluorescent material is too large, since the excitation efficiency of the fluorescent material is not 100%, the current to be applied to the LED elements 15 must be increased in order to ensure the total light intensity of the first light and the second light. Thus, the power consumption is inevitably increased.

It is preferable that the PDT light irradiation device of the present invention satisfies the following formula (1) where an irradiance integral value of light within a range of wavelengths of not shorter than 350 nm to not longer than 455 nm on the irradiated surface is defined as IA and an irradiance integral value of light within a range of longer than 455 nm to not longer than 650 nm on the irradiated surface is defined as IB.

IA/IB=0.2 to 5 Formula (1)

It is more preferable that the IA/IB in the above-described formula (1) is 1 to 1.8.

An absorbance of PpIX increases in order of a wavelength of 410 nm, a wavelength of 510 nm, a wavelength of 545 nm, a wavelength of 580 nm, and a wavelength of 630 nm. On the other hand, propagation of light of these wavelengths in the living body decreases in order of a wavelength of 410 nm, a wavelength of 510 nm, a wavelength of 545 nm, a wavelength of 580 nm, and a wavelength of 630 nm.

Thus, in a case where (IA+IB) has a constant value, if the value of IA/IB in the formula (1) is too small, that is, a relative irradiance of the second light with respect to the first light is too large, an effective irradiance acting on PpIX considering an absorbance becomes large in a part having a long distance from the living body surface in the lesion (hereinafter simply referred to as “deep lesion”). Conversely, the effective irradiance becomes small in a part having a short distance from the living body surface in the lesion (hereinafter simply referred to as “shallow lesion”). This causes a problem of reducing a therapeutic effect on the shallow lesion.

On the other hand, if the value of IA/IB in the formula (1) is too large, that is, a relative irradiance of the second light with respect to the first light is too small, the effective irradiance becomes large in the shallow lesion. Conversely, the effective irradiance becomes small in the deep lesion. This causes a problem of reducing a therapeutic effect on the deep lesion.

Further, a depth of the lesion varies. That is, the lesion sometimes exists only in a shallow part or in both shallow and deep parts. In a case where the lesion exits in a deep part invisible from the surface, a sufficient therapeutic effect may not be achieved after performing the photodynamic therapy due to growth of the remaining tumor. From such a viewpoint, setting IA/IB to 1 to 1.8 has an advantage of achieving a sufficient therapeutic effect on both the shallow lesion and the deep lesion.

In order to satisfy the above-described formula (1) in the fluorescent plate 20, the type of the fluorescent material, the content ratio of the fluorescent material, the thickness of the fluorescent plate and the like may be set as appropriate.

The contact member 25 is only required to have transparency. However, it is preferable that the contact member 25 can be elastically deformed according to a surface shape of the lesion as the irradiated surface to be brought into close contact with the lesion.

Further, it is preferable that the surface of the contact member 25 has an adhesive property to be in close contact with the lesion. The degree of the adhesive property on the surface of the contact member 25 is, for example, a vertical peel strength of 60 to 80 N as measured by a test performed under a condition of a tensile speed of 300 mm/min (hereinafter simply referred to as “vertical peel strength”).

Further, the fluorescent plate 20 is separated from the lesion surface as the irradiated surface by the contact member 25. The fluorescent material constituting the fluorescent plate 20 includes a metal material such as Ba and thus may cause metal allergy through direct contact with the living body, which is a problem.

Arranging the contact member 25 makes it possible to use the fluorescent plate 20 including the metal material causing metal allergy in a state of being separated from the lesion by the contact member 25. Thus, the PDT light irradiation device of the present invention can be applied to a patient with metal allergy.

It is preferable that the contact member 25 has a transmittance of not less than 80% for light emitted from the fluorescent plate 20 (mixed light of the first light and the second light).

As such a contact member 25, may be used a plastic bag processed so as to maintain a certain thickness and filled with water or air, an epoxy-based, polyurethane-based, or silicone-based transparent resin plate having flexibility, a water-absorbing polymer or styrene-based elastomer processed into a plate and members variously formed.

The above-described PDT light irradiation device is used in a state in which the contact member 25 is brought into close contact with a surface of the lesion. Specifically, when the contact member 25 in the PDT light irradiation device is brought into contact with and pressed against the surface of the lesion, the PDT light irradiation device having the flexible substrate 11 is deformed according to the surface of the lesion. This causes the contact member 25 in the PDT light irradiation device to be in close contact with the surface of the lesion therealong.

Then, when the PDT light irradiation device is operated, the first light having a peak wavelength within a range of wavelengths of not shorter than 400 nm to not longer than 420 nm is emitted from the LED elements 15 and made incident on a back surface of the fluorescent plate 20 via the protective resin layer 17. A part of the first light made incident on the fluorescent plate 20 passes the fluorescent plate 20 and is emitted from a surface of the fluorescent plate 20. Simultaneously, another part of the first light is absorbed by the fluorescent material in the fluorescent plate 20 and converted to the second light which is fluorescence having a wavelength of not shorter than 500 nm to not longer than 520 nm by the fluorescent material, and then the second light is emitted from the surface of the fluorescent plate 20. The first light and the second light emitted from the surface of the fluorescent plate 20 are superimposed and irradiated on the surface of the lesion as the irradiated surface via the contact member 25.

Such a PDT light irradiation device is deformed according to the surface of the lesion as the irradiated surface by having the flexible substrate 11 and thus makes it possible to keeps a constant distance between the irradiated surface and the LED element 15 although the surface of the lesion is formed in an uneven shape. Thus, it becomes possible to irradiate light with uniform illuminance to the irradiated surface.

Further, the first light transmitted through the fluorescent plate 20 and the second light converted by the fluorescent plate 20 are emitted from the surface of the fluorescent plate 20, and thus the first light and the second light are superimposed and irradiated on the surface of the lesion as the irradiated surface. Thus, it becomes possible to obtain a spectral distribution of high uniformity on the entire irradiated surface.

### EXAMPLES

While specific examples of the PDT light irradiation device of the present invention will be described below, the present invention is not limited to the following examples.

### Example 1

In accordance with the configuration in FIG. 1 and FIG. 2, the PDT light irradiation device having the following specifications were produced.

### [Light Source Unit]

Flexible substrate: material=liquid crystal polymer, size=35 mm×35 mm×50 μm

Light reflecting film: material=silver, thickness=5 μm, total light reflectance=92%

LED element: peak wavelength=404 nm, size=1 mm×1 mm×0.15 mm, total radiant flux=30 mW, number of LED elements=25 (arranged in a lattice pattern in five vertical rows and five horizontal rows), arrangement pitch=5 mm

Protective resin layer: material=silicone resin, thickness=0.8 mm

Wall material: material=silicone resin, external size=28 mm×28 mm×0.6 mm

### [Fluorescent Plate]

Transparent material: material=silicone resin

Fluorescent material: material=Ba₂SiO₄:Eu, content ratio of fluorescent material=4 mass parts per 100 mass parts of transparent resin

Size=28 mm×28 mm×1 mm

### [Contact Member]

Material=styrene-based elastomer, size=50 mm×50 mm×5 mm, hardness=Asker C15, surface adhesive property=vertical peel strength of 70 N

The above-described PDT light irradiation device is operated and an optical spectrum of light from the PDT light irradiation device on the irradiated surface right above the light source unit (hereinafter simply referred to as “irradiated surface”) was measured. As shown in FIG. 3, the light from the PDT light irradiation device includes the first light having a peak wavelength within a range of wavelengths of not shorter than 400 nm to not longer than 420 nm and the second light having a wavelength of not shorter than 500 nm to not longer than 520 nm. Without the fluorescent plate, only the first light is included, indicating that the second light is caused by fluorescence of the fluorescent plate.

Further, in the above-described PDT light irradiation device, the irradiance integral value IA of the light within a range of wavelengths of not shorter than 350 nm to not longer than 455 nm on the irradiated surface and the irradiance integral value IB of the light within a range of wavelengths of longer than 455 nm to not longer than 650 nm on the irradiated surface were measured to find that IA was 34.8 mW/cm², IB was 23.7 mW/cm², and the value of IA/IB was 1.47.

### Comparative Example 1

A PDT light irradiation device having the same configuration as that in Example 1 was produced except that the fluorescent plate was not used.

### Comparative Example 2

A PDT light irradiation device having the same configuration as that in Example 1 was produced except that the LED elements and the fluorescent material of the fluorescent plate were changed to those having the following specifications and a filter for cutting the light from the LED element was provided on the surface of the fluorescent plate.

LED element: peak wavelength=450 nm, size=1 mm×1 mm×0.15 mm, total radiant flux=20 mW

fluorescent material: K₂SiF₆:Mn (peak wavelength of fluorescence=635 nm)

### Comparative Example 3

A PDT light irradiation device having the same configuration as that in Example 1 was produced except that a rigid wiring substrate having the following specifications was used instead of the flexible substrate.

Wiring substrate: material=ceramic, size=35 mm×35 mm×500 μm

<Test>

A nude mouse of 4 to 6 weeks old having a weight of about 20 g was prepared and a solution of human melanoma cells (COLO679) (concentration: 2×10⁷ cells/mL) in an amount of 100 μL was inoculated by subcutaneous injection in the right and left shoulder joint parts of the mouse. A long diameter of a tumor in the lesion in this mouse was measured every two days. When the long diameter of the tumor in the mouse reached 5 to 7 mm (2 to 3 weeks after inoculation), the following agent was administered to the mouse. Subsequently, the mouse administered with the agent was left in a dark place for 4 hours.

Agent: δ-aminolevulinic acid (5-ALA) was used as a substance to be administered to the living body, the agent being prepared by dissolving the substance to be administered to the living body in an amount of 250 mg per kg weight of the mouse in a phosphate-buffered physiological saline solution.

Next, a light shielding sheet made of aluminum having an opening of 15 mm×15 mm was prepared, and the light shielding sheet was placed on the mouse such that the opening of the light shielding sheet is positioned on the lesion in the mouse. Then, each of the PDT light irradiation devices according to Example 1 and Comparative examples 1 to 3 is positioned such that the contact member is brought into contact with the lesion in the mouse, and each of the PDT light irradiation devices was pressed with a force of 1 N. Then, light was irradiated to the lesion in the mouse using each of the PDT light irradiation devices under conditions in which an illuminance, an irradiation time, and an irradiation amount on the irradiated surface caused by each of the PDT light irradiation devices were set to values shown in Table 1 below.

TABLE 1

| | | COMPAR- | COMPAR- | COMPAR- |
|---|---|---|---|---|
| | EXAM- | ATIVE | ATIVE | ATIVE |
| | PLE | EXAM- | EXAM- | EXAM- |
| | 1 | PLE 2 | PLE 3 | PLE 4 |
| ILLUMINANCE | 58.5 | 61.7 | 63.9 | 26.0 |
| (mW/cm²) | | | | |
| IRRADIATION | 855 | 810 | 782 | 1923 |
| TIME (sec) | | | | |
| IRRADIATION | 50 | 50 | 50 | 50 |
| AMOUNT (J/cm²) | | | | |

Then, an area of the tumor in the lesion was measured every 5 days in the mouse subjected to the light irradiation, and a relative value when an area of the tumor in the mouse immediately before the light irradiation was defined as 1 was obtained.

Since the surface shape of the tumor in the mouse had a substantially elliptical shape, the area of the tumor was calculated by the following formula (2):

Area of tumor=long diameter of tumor×short diameter of tumor×π Formula (2)

The results are shown in Table 2 below.

TABLE 2

| | AREA OF TUMOR IN MOUSE | | | |
|---|---|---|---|---|
| | (RELATIVE VALUE) | | | |
| | ON | ON | ON | ON |
| | DAY 5 | DAY 10 | DAY 15 | DAY 20 |
| EXAMPLE 1 | 0.0 | 0.0 | 0.3 | 0.5 |
| COMPARATIVE | 0.4 | 1.6 | 1.8 | 2.4 |
| EXAMPLE 1 | | | | |
| COMPARATIVE | 0.4 | 0.8 | 1.1 | 1.5 |
| EXAMPLE 2 | | | | |
| COMPARATIVE | 0.0 | 1.2 | 1.6 | 2.1 |
| EXAMPLE 3 | | | | |
| NO IRRADIATION | 1.7 | 3.8 | 4.9 | 6.7 |

The results in Table 2 confirmed that the tumor in the mouse disappeared 5 days after the light irradiation using the PDT light irradiation device according to Example 1. Further, the tumor in the mouse reappeared 15 days after the light irradiation; however, the area of the tumor was less than 1.0 and remained less than 1.0 up to 20 days after the light irradiation.

On the other hand, when the PDT light irradiation devices according to Comparative example 1 and Comparative example 2 were used, the area of the tumor in the mouse was not more than 0.5 five days after the light irradiation; however, the tumor in the mouse did not disappear. Further, the area of the tumor in the mouse increased 10 days after the light irradiation, and the area of the tumor in the mouse greatly exceeded 1.0 20 days after the light irradiation.

Further, when the PDT light irradiation device according to Comparative example 3 was used, the tumor in the mouse disappeared 5 days after the light irradiation; however, the tumor in the mouse reappeared 10 days after the light irradiation, and the area of the tumor also exceeded 1.0.

### REFERENCE SIGNS LIST

10 Light source unit
11 Flexible substrate
12, 13 wiring portion
15 LED element
16 Light reflecting film
17 Protective resin layer
18 Wall material
20 Fluorescent plate
25 Contact member

1. A photodynamic therapy light irradiation device comprising:
a light source unit having one or more LED elements that emit first light having a peak wavelength within a range of wavelengths of not shorter than 400 nm to not longer than 420 nm disposed on a flexible substrate; and
a fluorescent plate configured to transmit a part of the first light from the light source unit, and to convert another part thereof into second light having a wavelength of not shorter than 500 nm to not longer than 520 nm and thereby emit the second light.

2. The photodynamic therapy light irradiation device according to claim 1, wherein the light source unit includes a plurality of the LED elements.

3. The photodynamic therapy light irradiation device according to claim 1, wherein the fluorescent plate is disposed such that the first light and the second light are superimposed on an irradiated surface.

4. The photodynamic therapy light irradiation device according to claim 1, wherein the light irradiated from the fluorescent plate to the irradiated surface satisfies the following formula (1) where an irradiance integral value of light within a range of wavelengths of not shorter than 350 nm to not longer than 455 nm on the irradiated surface is defined as IA and an irradiance integral value of light within a range of longer than 455 nm to not longer than 650 nm on the irradiated surface is defined as IB:
IA/IB=0.2 to 5. Formula (1)

5. The photodynamic therapy light irradiation device according to claim 4, wherein the IA/IB in the formula (1) is 1 to 1.8.

6. The photodynamic therapy light irradiation device according to claim 1, wherein
the light source unit includes a wall material formed so as to surround a region where the LED element is disposed on the flexible substrate and a protective resin layer formed so as to cover the LED element in the region where the LED element is disposed, the region being surrounded by the wall material; and
the fluorescent plate is disposed so as to cover upper surfaces of the protective resin layer and the wall material.

7. The photodynamic therapy light irradiation device according to claim 6, wherein a contact member configured to have transparency and be brought into contact with the irradiated surface is provided so as to cover at least the fluorescent plate.

8. The photodynamic therapy light irradiation device according to claim 1, wherein the fluorescent plate includes Ba₂SiO₄:Eu as a fluorescent material.
